(19) United States
(12) Patent Application Publication
(10) Pub. No.: US 2006/0085048 A1
(43) Pub. Date: Apr. 20, 2006

(54) Algorithms for an active electrode, bioimpedance-based tissue discrimination system

(75) Inventors: Philip C. Cory, Bozeman, MT (US); Waldean A. Schulz, Boulder, CO (US); Joan M. Cory, Bozeman, MT (US); Mark Pernarowski, Bozeman, MT (US)

Correspondence Address: HELLER EHRMAN WHITE & MCAULIFFE LLP, 1717 RHODE ISLAND AVE, NW, WASHINGTON, DC, 20036-3001, US

(73) Assignee: Nervonix, Inc., Bozeman, MT (US)

(21) Appl. No.: 11/252,556

(22) Filed: Oct. 19, 2005

Related U.S. Application Data
(60) Provisional application No. 60/619,921, filed on Oct. 20, 2004.

(51) Int. Cl. A61N 1/08; A61B 5/05

(52) U.S. Cl. 607/48; 600/547

(57) ABSTRACT

A method and system for discriminating tissues in a subject, particularly for identifying nerve tissue, includes a processor, a waveform generator, a waveform electrode and a return electrode and an electrical property measuring device such as a volt meter. The electrodes are applied to the skin of the subject and an electrical waveform applied to the tissue via the electrodes. A series of electrical property measurements between the electrodes yield digital data that is used to determine coefficients of an approximating mathematical function, which is then used to derived electrical properties of the tissue. Derived electrical properties are then displayed on a display device.

### RELATED APPLICATIONS

This application claims the benefit of U.S. Provisional Patent Application No. 60/619,921, filed Oct. 11, 2004, the entire contents of which are incorporated herein in their entirety. This application is also related to U.S. application No. (to be issued) entitled “An Active Electrode, Bio-impedance Based, Tissue Discrimination System and Methods Of Use” filed concurrently herewith, the entire contents of which are hereby incorporated by reference. This application is also related to U.S. Patent Application No. 20030009111, the entire contents of which are hereby incorporated by reference.

### FEDERALLY SPONSORED RESEARCH

Not applicable

### FIELD OF INVENTION

This invention relates to the discrimination and mapping of biological tissue types, and more particularly to the discrimination and mapping of peripheral nerve tissue using non-invasive electrodes and applied electric fields to determine certain electrical characteristics or properties of tissues of living subjects and using these characteristics to determine other tissue features and locations.

### BACKGROUND

Non-invasive means of detecting subcutaneous tissue have been of interest to scientists and clinicians for hundreds of years, but only during the twentieth century have several high energy technologies been developed for this purpose. These include x-radiography, nuclear magnetic resonance imaging, and ultrasound. Additional, minimally invasive technologies employing radioisotopes are also used for such tissue discrimination activities as positron emission tomography and radionuclide scanning. Low energy approaches have been limited to skin surface application of low intensity electrical fields, which enable measuring the developed skin surface potentials and applying back projection algorithms to reconstruct the tissues' effect on the electrical field path, e.g., electrical impedance tomography (EIT).

Recently, a non-invasive tissue detection technology has been developed by Cory and disclosed in U.S. Pat. No. 5,560,372 (which is hereby incorporated by reference in its entirety) based on the finding that nerves are detectable using very low intensity electrical fields to determine low impedance sites on the skin. The ability to detect nerves as low impedance sites depends on the presence of electrically responsive elements embedded in biologic, lipid bilayer, membrane structures coupled with the ability of long, uninterrupted, electrolyte-filled tubes (axons) to electrotonically conduct applied electrical fields. Importantly, this nerve detection ability occurs at electrical field intensities that are subthreshold, i.e., below the strength required to depolarize an axonal cell membrane to the point of propagated action potential generation. This finding was distinct from the known impedance changes observed in nerves at suprathreshold electrical field strengths (approximately 100 mV/cm at the axonal cell membrane; Cooper, 1995) during depolarization and action potential propagation.

Evaluations of tissue with the impedance measurement technology of U.S. Pat. No. 5,560,372 revealed that the magnitude of differences in electrical characteristics determined from measurements on the skin correlated with the amount of exposed neuronal cell membrane expected in the underlying tissue and, consequently, with the expected density of voltage-gated ion channels in underlying tissue, i.e., neuromas have the highest known density of voltage-gated channels per gram of tissue and demonstrate the greatest magnitude change, followed in decreasing order by nerve entrapments, nerve contusions, and normal nerve tissue. Features of normal neuroanatomy also correlate with the magnitude of impedance change observed using this technology. Nerve branch points, for example, exhibit greater impedance changes (e.g., lower impedance) than does nerve tissue without a major branch point. Such branch points likely are responsible for some of the biologically active point (BAP) observations discussed in the acupuncture literature. Evaluation of acupuncture points with this technology reveals that these sites were frequently associated with nerve branching, whether normal, parallel, or oblique to the plane of the skin surface. Similarly, this technology demonstrated that myofascial trigger points were associated with nerve entrapments at the deep myofascial boundary where branches of underlying mixed-function nerves, normal to the plane of the skin surface, penetrate through the fascial investment of the muscle. Myofascial trigger point formation thus couples an abnormality (nerve entrapment) with a normal, anatomic structure (a nerve branch).

The technology of U.S. Pat. No. 5,560,372 is based upon the recognition that nerves present a preferential conduction pathway for subthreshold electrical fields, i.e., electrical fields of insufficient amplitude to generate action potentials. A key factor in these observations was repeated demonstration that nerves and their associated abnormalities, detected at the skin surface, occur along a normal to the complex surface of the skin, which intersects the nerve structure at depth. This relationship has been verified to depths of over 8 cm with target structures in the 1-2 mm range.

The recognition that tissue represents a non-homogeneous conductor best modeled as a parallel resistance and capacitance with a series resistance has enabled determination of the bulk conductor electrical properties of tissue. Below are listed notable research papers in this field establishing some of the physiological and technological foundation upon which the present invention is based:
1. Oaklander A L: The Density of Remaining Nerve Endings in Human Skin with and without Postherpetic Neuralgia after Shingles. Pain 2001; 92: 139-45;
2. McArthur J C, Stocks E A, Hauer P, Cornblath D R, Griffin J W: Epidermal Nerve Fiber Density. Arch. Neurol. 1998; 55: 1513-20;
3. Petersen K L, Rice F L, Suess F, Berro M, Rowbotham M C: Relief of post-herpetic neuralgia by surgical removal of painful skin. Pain 2002; 98: 119-26;
4. Nolano M, Simone D A, Wendelschafer-Crabb G, Johnson T, Hazen E, Kennedy W R: Topical capsaicin in humans: parallel loss of epidermal nerve fibers and pain sensation. Pain 1999; 135-45;
5. Hodgkin A L, Huxley A F: A Quantitative Description of Membrane Current and its Application to Conduction and Excitation in Nerve. J. Physiol. 1952; 117: 500-44;
6. Rall W: Core Conductor Theory and Cable Properties of Neurons, Handbook of Physiology, section 1, The Nervous System. Edited by Brookhart J M, Mountcastle V B, Kandel E R. Baltimore, Md., Baltimore, Md., 1977, pp. 39-97;
7. Finkelstien A, Mauro A: Physical Principles and Formalisms of Electrical Excitability, The Nervous System. Edited by Brookhart J M, Mountcastle V B, Kandel E R. Baltimore, Md., Waverly Press, Inc, 1977, pp. 161-213;
8. Mauro A: Anomalous Impedance, A Phenomenological Property of Time-Variant Resistance: An Analytic Review. Biophysical Journal 1961; 1: 353-72;
9. Cooper M S: Membrane Potential Perturbations Induced in Tissue Cells by Pulsed Electric Fields. Bioelectromagnetics 1995; 16: 255-62;
10. Sabah N H, Leibovic K N: Subthreshold oscillatory responses of the Hodgkin-Huxley cable model for the squid giant axon. Biophys. J. 1969; 9: 1206-22;
11. Mauro A, Conti F, Dodge F, Schor R: Subthreshold behavior and phenomenological impedance of the squid giant axon. J. Gen. Physiol. 1970; 55: 497-523;
12. Cole K S, Baker R F: Longitudinal impedance of the squid giant axon. J. Gen. Physiol. 1941; 24: 771-88;
13. Cole K S: Rectification and inductance in the squid giant axon. J. Gen. Physiol. 1941; 25: 29-51;
14. Rudy Y, Plonsey R: The eccentric spheres model as the basis for a study of the role of geometry and inhomogeneities in electrocardiography. IEEE Trans. Biomed. Eng. 1979; BME-26: 392-9;
15. Cole K S: Electric impedance of suspensions of spheres. J. Gen. Physiol. 1928; 12: 29-36;
16. Cole K S: Electric impedance of suspensions of arbacia eggs. J. Gen. Physiol. 1928; 12: 37-54;
17. Cole K S: Electric phase angle of cell membranes. J. Gen. Physiol. 1932; 15: 641-9;
18. Cole K S, Hodgkin A L: Membrane and protoplasm resistance in the squid giant axon. J. Gen. Physiol. 1939; 22: 671-87;
19. Cole K S, Baker R F: Transverse impedance of the squid giant axon during current flow. J. Gen. Physiol. 1941; 24: 535-49;
20. Cole K S: Membranes, ions, and impulses. Berkeley and Los Angeles, University of California Press, 1972, pp. 1-569;
21. Cooper M S: Gap junctions increase the sensitivity of tissue cells to exogenous electric fields. J. Theor. Biol. 1984; 111: 123-30;
22. Gabriel C, Gabriel S, Corthout E: The dielectric properties of biological tissues: I. Literature survey. Phys. Med. Biol. 1996; 41: 2231-49;
23. Gabriel S, Lau R W, Gabriel C: The dielectric properties of biological tissues: II. Measurements in the frequency range 10 Hz to 20 GHz. Phys. Med. Biol. 1996; 41: 2251 -69;
24. Gabriel S, Lau R W, Gabriel C: The dielectric properties of biological tissues: III. Parametric models for the dielectric spectrum of tissues. Phys. Med. Biol. 1996; 41: 2271-93;
25. Rall W: Theory of Physiological Properties of Dendrites. Ann. NY Acad. Sci. 1962; 96: 1071-92;
26. Holder D S: Impedance changes during the compound nerve action potential: implications for impedance imaging of neuronal depolarisation in the brain. Med. & Biol. Eng. & Comput. 1992; 30: 140-6;
27. Jongschaap H C N, Wytch R, Hutchison J M S, Kulkarni V: Electrical Impedance Tomography: A Review of Current Literature. Eur. J. Radiol. 1994; 18: 165-74;
28. Kwok G, Cohen M, Cosic I: Mapping Acupuncture Points Using Multi Channel Device. Australas. Phys. Eng. Sci. Med. 1998; 21: 68-72;
29. Lykken D T: Square-Wave Analysis of Skin Impedance. Psychophysiology 1971; 7: 262-75;
30. Kaslow A L, Lowenschuss O: Dragon Chasing: A New Technique for Acupuncture Point Finding and Stimulation. Am. J. Acupunct. 1975; 3: 157-60;
31. Reichmanis M, Marino A A, Becker R O: Electrical Correlates of Acupuncture Points. IEEE Trans. Biomed. Eng. 1975; BME 22: 533-532;
32. Johng H M, Cho J H, Shin H S, Soh K S, Koo T H, Choi S Y, Koo H S, Park M S: Frequency Dependence of Impedances at the Acupuncture Point QUZE (PC3). IEEE Eng. Med. Biol. 2002; 33-6;
33. Prokhovav E, Llamas F, Morales-Sanchez E, Gonzalez-Hemandez J, Prokhorav A: In Vivo Impedance Measurements on Nerves and Surrounding Skeletal Muscles in Rats and Human Body. Med. & Biol. Eng. & Comput. 2002; 40: 323-6; and
34. Geddes L A: Historical Evolution of Circuit Models for the Electrode-Electrolyte Interface. Ann Biomed Eng 1997; 25: 1-14.

### SUMMARY OF THE INVENTION

The present invention provides improved algorithms for obtaining clinically meaningful information from electrical characteristics or properties, particularly impedance, measured by electrodes on a surface of a body—e.g., electrodes positioned on the skin—in the presence of an applied electrical field.

The present invention includes both systems and methods for discriminating tissue. The system includes some or all of a programmable processor, a waveform generator configured to generate an applied waveform at least one property of which is controlled by the processor, a waveform electrode and a return electrode electrically connected to the waveform generator and suitable for application to the skin of a person, a measurement circuit connected to the processor, the waveform electrode and the return electrode and configured to measure at least one electrical attribute (e.g., voltage or current) between the electrodes; and a display connected to the processor. The processor in the system is programmed to carry out the steps of the methods of the present invention, which includes some or all of the steps of specifying parameters to the waveform generator describing at least one waveform and specifying whether the waveform relates to an applied voltage or an applied current, directing the waveform generator to generate at least one repetition of the waveform which is applied across the waveform electrode and the return electrode when each is positioned on the skin of a person, receiving a temporally discrete sequence of samples of at least one electrical property measured between the waveform electrode and the return electrode, saving the received samples as a sequence of digital numbers, using the sequence of digital numbers, calculating parameters characterizing a mathematical function having time as an independent variable so that the mathematical function approximates the sequence of digital values at times associate with each value, deriving electrical properties from the parameters characterizing the mathematical function, and presenting the derived electrical property values in a human understandable form. The waveform parameters may be amplitude frequency, wave shape (e.g., square or rectangular wave, sinusoidal wave, etc.) and duration (e.g., number of cycles) of the waveform signal.

### DESCRIPTION OF THE DRAWINGS

The accompanying drawings, which are incorporated herein and form a part of the specification, illustrate a preferred embodiment of the present invention and, together with the description, serve to explain the principle of the invention.

FIG. 1 is a depiction of the distribution of an electric field in a homogeneous, bulk conductor containing an ovoid region of increased conductivity and a nerve.

FIG. 2 is a depiction of the distribution of an electric field in a homogenous, bulk conductor containing a nerve with axons extending to the skin surface where the nerve and axons represent electrical anisotropicities in the conductor.

FIG. 3 is a schematic diagram of the hardware of the present invention.

FIG. 4. is a flow diagram of a method of the present invention.

FIG. 5 is a simplified schematic diagram showing the electrical circuit equivalent in principle to the electronics of the present invention.

FIG. 6 is a simplified schematic diagram showing the electrical circuit equivalent in principle to the electronics of the present invention with a wireless connection to the microprocessor.

FIG. 7 is a screen shot of prototype of a system embodying the present invention with an accompanying MRI scan of the region of the screen shot.

FIG. 8 is a plot of an example signal received by an embodiment of the present invention in the presence of an example applied waveform signal.

### DETAILED DESCRIPTION

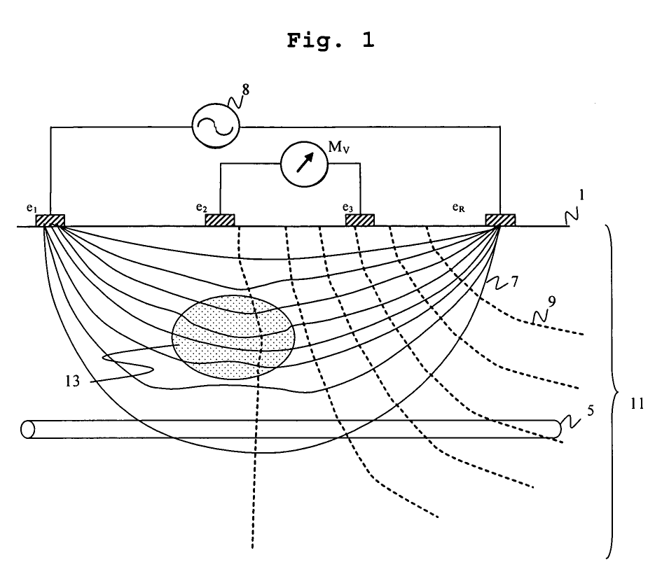

In FIG. 1, the standard depiction of an electrical field following a prolate ellipsoid course through a bulk conductor is shown. The electric field generator 8 applies a current between electrodes e₁ and e_{R} located on the skin surface 1 of the tissue bulk conductor 11. Contained within the nonhomogenous conductor is a region of increased conductance 13 that causes the isocurrent lines 7 to bend leading to a variation in the skin surface position of the equipotential lines 9. The voltmeter M_{V} detects the potential difference between electrodes e₂ and e₃. The nerve 5 is classically thought to present such a small conductance variation as to be not relevant in deflecting the equipotential lines at the skin surface; this classic representation is inconsistent with observations made using the present invention.

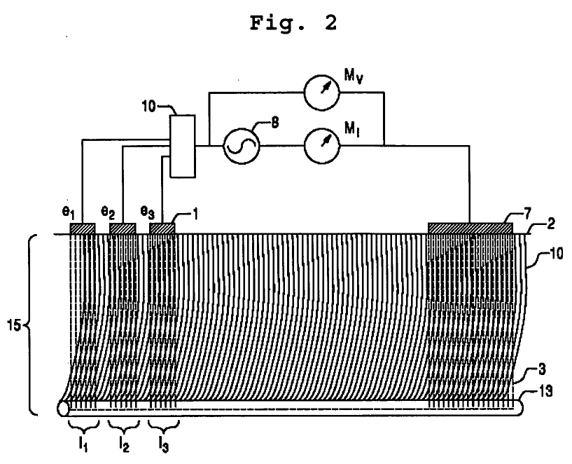

In FIG. 2, a more accurate depiction of the nerve 5 and its associated axons 3 as electrical anisotropicities within the tissue conductor 2 is presented. Current is supplied by the generator 8 to the switching device 10 that directs the current to electrodes e₁, e₂, and e₃. The isocurrent lines 7 cross the skin surface 1 and are directed into the interior of the axons 3 and hence along the nerve 5 following a right angle relationship to the skin surface 1 and the return electrode e_{R}. The applied voltage required to drive the current at each electrode may be measured by voltmeter M_{V}. The voltages detected by voltmeter M_{V} reflect the impedances measured along the segment of nerve 5 at i₁, i₂, and i₃. Back projection algorithms based on non-homogeneous, isotropic tissue models suffer from the significant error source of neglecting the anisotropicity associated with nerves and axons in living tissue. Such back projection algorithms attempt to model the equipotential lines, shown in FIG. 1, occurring along the course of an applied electrical field coursing between two skin surface points to determine the nature of the underlying tissue electrical characteristics or properties. Although it has been presumed that at relatively high frequencies (into the megahertz range) electrical fields transit the body through the extracellular fluid space, this is clearly questionable in light of the Prokhorov data (paper 33 listed above). Consequently, the use of a classic model based on Maxwell's equations for an isotropic, non-homogeneous conductor will not lead to an accurate description of those underlying tissues unless the anisotropicity of living tissue is also included in the model.

The present invention provides an improved apparatus and method for accurately locating and discriminating subcutaneous tissue structures. This method modifies the tissue electrical model that includes the non-homogeneities of different tissue subtypes to account for the observation that certain types of tissue, particularly nerve tissue, present preferential pathways through tissue for applied, subthreshold electrical fields, even at megahertz (MHz) frequencies. The preferential pathways presented by nerve tissue comprise a high density collector system in the dermal tissues leading into a long, uninterrupted, conduction pathway that is highly parallel and exhibits a large capacitance relative to non-nerve structures. Associated with this collector and conduction system is a right-angle relationship from the skin surface to underlying nerve structures that is most likely a result of the anatomic relationships of nerves to the surrounding tissue. Individual axons are best modeled as leaky, one dimensional cables which maintain the majority of the applied field intra-axonally, but allow some portion of the applied field to transit the surrounding tissue between axons or within a nerve bundle. Though the axoplasm demonstrates a bulk resistivity that is similar in magnitude to that of the extracellular fluid, the interior of axons lacks conduction barriers such as those presented by cell membranes in the surrounding tissue. An applied electrical field may travel in the extracellular fluid medium, but it will encounter these tissue barriers [represented as resistances and capacitances (RC) in series and in parallel] whereas the interior of the axon presents an ohmic resistance without the RC barriers. Furthermore, there is a large capacitance associated with axon structure as a consequence of the long, cylindrical form of the nerve cell. Since the lipid bilayer structure of the cell membrane has a capacitance of approximately 1 μF/cm², the long cylindrical structure of the single axon has a much greater associated capacitance than any other, geometrically discrete, cell type. Additionally, axons travel in bundles as nerves. The result is a highly parallel capacitance and resistance structure where the total resistance is the reciprocal of the sum of the reciprocal individual resistances, and the total capacitance will be the sum of the individual capacitances. This means that as the total number of axons within a nerve bundle increases, the total resistance is expected to fall asymptotically while the total capacitance progressively rises. Since impedance is directly related to resistance and inversely related to capacitance, the net result is a large fall in impedance associated with nerve structures.

The impedance relationship observed using the present invention is distinct from that observed with action potential propagation in nerves. When nerves conduct propagated action potentials, the nerve associated impedance falls as a result of increased conductivity due to opening of voltage-gated ion channels. This action potential associated impedance change is also time variant, reflecting the activation and inactivation functions of the voltage-gated sodium channels in response to a single, depolarizing pulse.

The recognition that a significant capacitive component plays a role in the preferential conduction of electrical fields along axons implies that frequency relationships are important. Research shows that a best frequency for discriminating nerve-rich structures from nerve-poor structures occurs in the 0.5-2 kHz range, although the nerve-related electrical parameter changes have been observed over a range of frequencies from 10⁰ Hz to 10⁶ Hz. This best frequency range is well below that associated with ionic or dipole relaxation frequencies associated with changes in permittivity. Molecular processes associated with ion channel gating mechanisms (i.e., activation and inactivation functions of the sodium channel) occur over time periods in the best frequency range, and may partially account for the effect.

The technology of the present invention reveals that current flow in living tissue does not conform to previous models for tissue impedance measurements. The model of electrical field distribution through bulk tissue that is used in electrical impedance tomography (EIT) or for functional electrical stimulation (FES) derives from theoretical current flow calculations for bulk conductors. These calculations start with the application of Maxwell's equations in homogeneous, bulk conductors and are modified to account for non-homogeneities within the bulk conductors, which represent tissues of varying resistivities. Similar approaches have been used for over a century in resistivity prospecting whereby underground ore bodies are identified through surface resistance mapping. Although complex back projection algorithms have been developed to create images of constituent tissues lying in an electrical field, the resolution of these images continues to be inadequate for routine clinical use. The underlying problem for these back projection algorithms is that tissue is not only non-homogeneous, it is also anisotropic. The most remarkable, and heretofore unrecognized, anisotropic feature of living tissue is that the neuroanatomy represents preferential conductance pathways through tissue, altering current flow from a prolate ellipsoid shape to a more constrained and angular path following the major nerves. To provide a more valid model for EIT and FES use, the nerve density and depth information beneath an electrode array must be taken into account. After mapping the anatomic distribution of the major conductive pathways (nerves), a model for electrical field distribution can then be constructed and the predictive distributions of skin surface potential determined for comparison with the actual distributions. A relevant example comprises the observations upon which some EIT breast cancer detection systems are based. Breast cancer lesions are reported to exhibit significantly higher resistance values and lower capacitance values than normal tissue or benign tumors. These observations are consistent with a paucity of nerve tissue in non-neural malignancies. Useful information can therefore be obtained using the present invention to determine not only where nerves exist, but also where nerves are expected and not present at normal levels.

The impedance differences associated with nerve tissue are observed with both the bipolar electrode system of the present invention and with tetrapolar electrode arrangements such as those used by Prokhorov. Though tetrapolar systems demonstrate lower impedances when used at the skin surface, supposedly by eliminating the high impedance of the stratum corneum, it is not clear that this impedance adjustment is necessary or desirable, particularly with the large site-to-site impedance differences observed using the technology of the present invention.

A theoretical problem exists with the tetrapolar electrode arrangements in that, with a current distribution model revealed by the present invention, the classic assumptions regarding the distribution of potential on the skin surface are inaccurate. Assuming a smooth, prolate ellipsoid distribution of current flow through a bulk conductor leads to the prediction that such electrical fields will be associated with a smooth distribution of equipotential lines on the conductor surface and that the distribution of surface potential may be directly related to the underlying total current flow. With an anisotropicity that dictates a right angle relationship, the surface potential distribution will not be smooth, but will demonstrate discontinuities (e.g., large variations compared to nearby sites), particularly in regions where the current flow transitions from a track coursing normal to the surface to a track coursing roughly parallel to the surface. This transition will be reflected by more closely spaced equipotential lines on the surface at some distance from the current carrying electrodes and will be related to the preferential conductance pathway depth. As a consequence, surface potential measurements performed between the current carrying electrodes will demonstrate variability that is most marked in these transition regions.

The technology of the present invention also demonstrates that sampling small skin surface areas is desirable because such small sites more accurately reveal the surface distribution of tissue impedance variation, which is a direct demonstration of the underlying anatomy. Electrodes such as those most often used in EIT, often sample larger areas (EIT typically employs ECG electrodes which are square with sides of 1.5 cm or circular with diameters of 1.5 cm, and thus range in area from about 1.8 cm² to about 2.25 cm²), thereby integrating the small area impedance variations and losing the site to site impedance variability seen using the present invention. In addition, for any given electrode size, tetrapolar electrode arrangements necessarily sample twice the surface area than do corresponding bipolar electrode systems. Consequently, for sampling electrodes of the same diameter, site to site discrimination will always be lower with tetrapolar systems.

From the foregoing, both tetrapolar and bipolar electrode systems may be used for tissue discrimination and nerve detection. However, bipolar electrode arrangements are less subject to potential discontinuities at the skin surface (e.g., large variations compared to nearby sites), and demonstrate better ability to discriminate tissues, particularly nerve tissue.

An important result of the present invention is the recognition that applying electrical fields using a controlled voltage approach represents a marked improvement in discrimination over the use of controlled current. This is particularly so when the output signals of the apparatus are multiplexed to different electrodes in an array of electrodes in the course of a measurement session. The use of controlled current outputs in a multiplexed device results in a lower applied voltage when the signal is multiplexed to an electrode overlying nerve-rich tissue because of the lower associated impedance at that site. The voltage variability resulting from electrode to electrode impedance variations disadvantageously leads to variation in the responses of voltage dependent cell membrane processes with concomitant reduction in measurement differences from site to site. Such disadvantages are avoided by controlling the voltage of the electric field applied to each electrode.

Voltage-gated ion channels in axonal cell membranes respond to time variant voltage gradients. This was first described mathematically by Hodgkin and Huxley in their landmark 1952 paper (paper no. 5 in the foregoing list). Importantly, these membrane proteins do not respond to time varying current except as they are exposed to the associated voltage gradient of that current. Since the present invention identifies nerves and their abnormalities in a fashion that appears related to the tissue densities of sodium channels, it is very possible that these channels play a role in both the resistive and the reactive components of the tissue impedance. The use of a controlled voltage system exposes all of the multiplexed electrodes in the apparatus to the same voltage and creates the same electrical intensity between the current carrying electrodes. If voltage-gated channels contribute to the technology of the present invention, then the effect may be optimized by maintaining the same voltage output even over low impedance electrodes. Consequently, for calculation of tissue electrical parameters it is preferable to measure the output current waveform, cyclic or acyclic, while controlling the output voltage. Also contemplated as part of the present invention is the option of simultaneously measuring the applied voltage and the applied current.

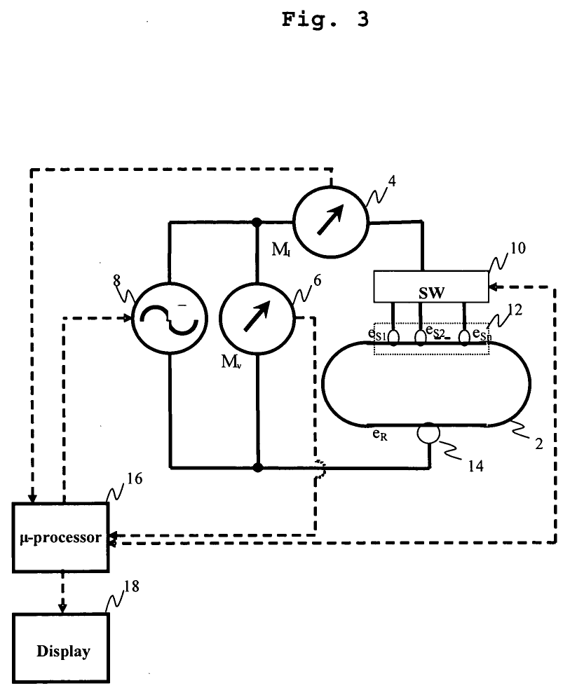

A suitable system to acquire and process the measurements for the present invention is illustrated in FIG. 3. In general, this apparatus may comprise the following: a waveform generator 8 configured to generate at least one periodic waveform in response to instructions received from a processor, such as a microprocessor 16, microcomputer, or microcontroller; at least one pair of spaced-apart electrodes 12, 14 operable to apply a waveform (i.e., the applied waveform) to tissue 2 of the subject; and at least one circuit 6 to measure attributes (e.g., voltage, current, or other electrical property) of a waveform measured across a tissue path (i.e., the measured waveform) which reveal the effects of the tissue on the applied waveform; a processor, such as a microprocessor 16 which receives the measurement information regarding the measured waveform and calculates at least one electrical characteristic (e.g., impedance, admittance, reactance, resistance, capacitance, inductance, etc.) of the tissue of the subject; software or circuitry to implement at least one alternative calculation method described below; and a display, such as a CRT screen 18, LCD, plasma, or other display technology, to display the result(s) of the calculation. This system is described in more detail below, followed by a description of suitable calculation methods, i.e., algorithms.

Digital methods have been chosen for use in describing the invention in this application for their simplicity. It is recognized that analog or digital-analog hybrid approaches, such as the use of analog circuit elements in place of digital calculations, could also be used.

In the apparatus illustrated in FIG. 3, the microprocessor 16 and associated circuitry is operable to control the waveform generator 8 to generate at least one electrical waveform to be applied to the tissue between at least two electrodes, to sample one or more electrical attributes (e.g., voltage or current) of the measured waveform (i.e., the waveform or signal measured between the two electrodes), and to calculate from the sampled electrical attributes an electrical characteristic (e.g., impedance, admittance, or other electrical characteristic) of the tissue between the electrodes for each generated and applied waveform (as above). Another example of a measured or calculated electrical characteristic is the phase relationship (e.g., phase lag) between an applied voltage waveform and the resulting current waveform. The waveform may be a sinusoidal wave, a rectangular wave, some other periodic wave, a constant non-zero amplitude waveform, a single impulse, some other aperiodic waveform, or some additive combination thereof. One preferred waveform (herein called a monophasic sinusoidal waveform) is the combination of a sinusoidal waveform plus a constant offset level resulting in entirely non-negative current or voltage amplitudes throughout the waveform.

In the apparatus of the present invention, the waveform electrode 12 may comprise a plurality of waveform electrodes eₛ₁, eₛ₂ . . . eₛₙ and the apparatus may further comprise a switching device or multiplexer 10 operable to receive instructions from the microprocessor 16 to apply a waveform between any one waveform electrode eₛ₁, eₛ₂ . . . eₛₙ of the plurality of waveform electrodes 12 and a return electrode 14. Alternatively, the switching device or multiplexer 10 may be operable to simultaneously provide a single waveform to more than one waveform electrode eₛ₁, eₛ₂ . . . eₛₙ.

FIG. 3 shows the plurality of waveform electrodes eₛ₁ through eₛₙ mounted on a substrate to form an electrode array 12, which is positioned in contact with the tissue of the subject 2. The electrode array 12 may be a geometrically arranged array of waveform electrodes, such as disclosed in U.S. Pat. Nos. 6,564,079 and 6,609,018, both of which are incorporated herein by reference in their entireties. FIG. 3 also shows one return electrode 14. Similar to the waveform electrodes, the return electrode 14 may comprise a plurality of return electrodes and its own switching device (not shown) that controls to which of the plurality of return electrodes the waveform is applied. A waveform generator 8 applies a waveform of specified parameters between at least one electrode eₛᵢ and at least one return electrode 14. The waveform electrode(s) to which the waveform is applied at any one time may be controlled by multiplexer 10. If there is more than one return electrode, the same or an additional multiplexer (not shown) would control which return electrodes are active.

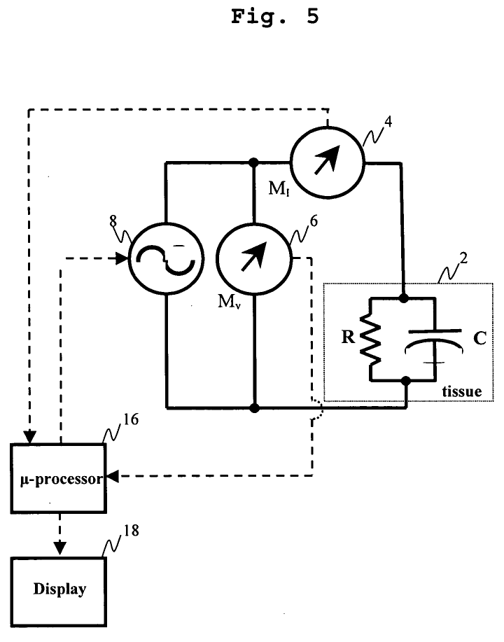

In operation, the processor, such as microprocessor 16 passes waveform generation specifications (such as frequency, amplitude, shape, DC offset, and so forth) to the waveform generator 8. The waveform generator may generate either a voltage waveform or a current waveform conforming to those specifications. The waveform need not be a simple, non-varying waveform; rather there may be reasons to implement complex waveforms or multiple waveforms; e.g., enhanced resolution of complex impedance models. The microprocessor 16 and/or switch 10 also implements the sampling, measurement, and digitization of a sequence of numeric measurements of at least one electrical characteristic (such as current flow), which is a function of the applied waveform (such as a controlled voltage waveform) and of a local electrical characteristic of the tissue volume to which the waveform is applied, as well as directing the storage of measurement data in memory. As discussed above, the electrical response of tissue, particularly tissue including nerve, may be modeled as a parallel resistor capacitor (RC) circuit element, as illustrated in FIG. 5. Using this approximation, the microprocessor 16 may sample electrical measurements (i.e., current and/or voltage) and perform calculations upon the data in the same manner as would be used to characterize a parallel RC circuit like that illustrated in FIG. 5. Microprocessor 16 may implement the majority of the computation steps in the analysis algorithm described in detail herein.

The microprocessor 16 may be any type of computing device. The present invention is not limited to an apparatus employing a microprocessor, and references herein to a microprocessor are meant to encompass all forms and configurations of processors, including microcomputers, microcontrollers, microprocessors, and computers, including external computers and workstations that may be programmed by software to accomplish tasks, such as to direct the functioning of attached electronics, receive, process and store data, perform analytical computations upon received data, communicate with external processors, generate displays and communicate via networks. In an embodiment, the microprocessor 16 is programmed with software that allows the microprocessor to receive commands from an operator to define the parameters of the waveform, e.g., the shape of the waveform, the positive and negative peak amplitudes, the frequency, and the duty cycle. The microprocessor 16 may be coupled to a memory, such as a volatile or nonvolatile memory, or to a computer readable medium device (e.g., compact disc reader, floppy disc, hard disc, magnetic tape, or other similar storage medium and reader) for storing and receiving software instructions for implementing the methods and algorithms described herein. The microprocessor 16 may also contain or be coupled to a memory bank having a plurality of predefined waveforms and may select waveforms to be generated by the waveform generator from the predefined set of waveforms. Microprocessor 16 may alternatively be configured to receive commands from a controller (e.g., a personal computer, not shown) electronically connected to the microprocessor 16, e.g., by a digital data link as known in the art (e.g., Fire Wire, USB, serial or parallel interface, etc.), or by means of a wireless data link as well known in the art.

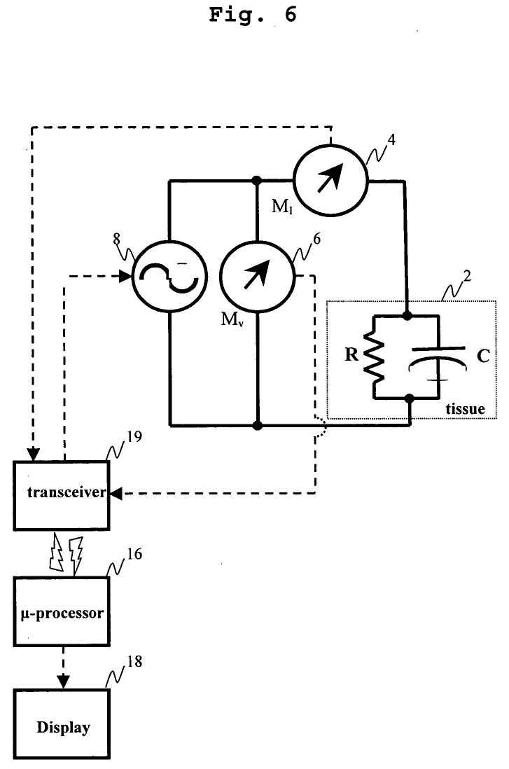

In another embodiment illustrated in FIG. 6, the controlling microprocessor 16 may be physically separated from the waveform generator 8, voltage meter, and electrodes but coupled to these elements by means of a transceiver 19. The transceiver 19 may communicate with the microprocessor 16 by any of a number of wired and wireless communication means well known in the art, including for example, Fire Wire, USB, serial or parallel interface, WiFi, Bluetooth, infrared, or other electronic communication protocol that may be developed. Separating the controlling processor 16 and display 18 from the rest of the equipment may provide a number of advantages, including facilitating the positioning of equipment in an examination room, and enabling sterilization of equipment that comes in contact with patients.

In another embodiment, the microprocessor 16 may generate the waveform, in which case an amplifier or array of amplifiers may be provided between the microprocessor 16 and the waveform generator circuit 8 to boost the signal (e.g., voltage or current) to the desired value, which may be controlled by the microprocessor 16.

In an embodiment of the present invention, a known and controlled voltage waveform is applied to the tissue, while the resulting current flow is sampled, measured, and processed to calculate at least one characteristic of the tissue, such as impedance or admittance. In another embodiment of the present invention, a known and controlled current waveform is applied to the tissue, while the required voltage necessary to generate the specified current flow is measured, sampled, and processed to calculate at least one characteristic of the tissue. In another embodiment of the present invention, the applied current waveform and the applied voltage waveform are measured, sampled, and processed simultaneously to calculate at least one characteristic of the tissue. In yet another embodiment of the present invention, the applied current waveform is controlled and the applied and measured voltage waveforms are sampled and processed simultaneously to calculate at least one characteristic of the tissue. As used herein, the different qualities that may be measured in each sample between the electrodes, such as current or voltage, are referred to herein collectively as electrical attributes, which may be constant or time varying properties, depending upon the applied waveform.

In an embodiment of the present invention, where each waveform electrode is a member of a geometrically arranged array of waveform electrodes, a plurality of waveforms may be applied simultaneously to more than one electrode in a manner which provides the same or different waveform to each of the electrodes of the array of electrodes.

The preferred ranges for controlled currents and controlled voltages that may be applied are as follows. Operating in constant current mode, the current applied to the waveform electrode may be between the minimally achievable and approximately 300 μA, more preferably up to approximately 100 μA. Similarly, in the controlled voltage mode, the current that is permitted to pass through tissues will be limited to between the minimally achievable and approximately 300 μA, more preferably up to approximately 100 μA. The amplitude of a time varying applied signal will range from the minimal achievable and approximately 24 V, more preferably up to approximately 5 V in the controlled current mode, and from the minimally achievable and approximately 300 μA, more preferably up to approximately 100 μA in the controlled voltage mode. The frequency of a time varying applied signal ranges from approximately 1 Hertz (Hz) to approximately 10 kilohertz (kHz), and more preferably between approximately 0.5 kHz and approximately 2.5 kHz. The waveform of the applied signal may be any wave shape, and more preferably may be any one or a combination of a monophasic or a biphasic, sinusoidal or square waveform. In the controlled current mode, measurements of the voltage waveform may be made immediately upon applying the current waveform or at any time thereafter. In the controlled voltage mode, measurements of the current waveform may be made immediately upon applying the voltage waveform or at any time thereafter. Measurement durations may range from approximately 10⁻⁵ seconds to approximately 1 second per each electrode, and more preferably approximately 0.01 seconds per each electrode.

The apparatus of the present invention may further comprise a display 18, and the microprocessor may generate a table, graph, or image on the display of the measured or calculated properties for some or all electrodes present. The values may be displayed numerically, graphically, or both. The values may be geometrically laid out in positions on the display that correspond to the geometrical locations of the electrodes, may simply appear in a list, or the electrode with the lowest impedance may be identified. Furthermore, such information may be overlaid on a picture or diagram of the electrode(s) and the substrate (if any) on which the electrodes reside.

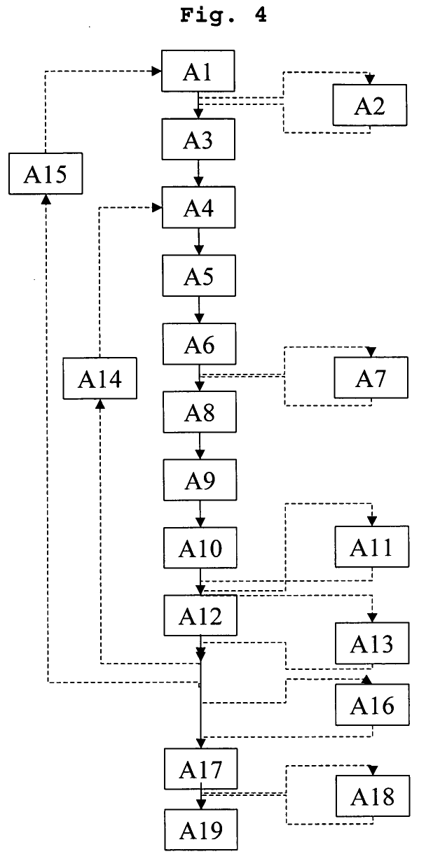

According to one embodiment of the present invention, the apparatus described above may be obtained by performing the following algorithmic steps, as illustrated in FIG. 4:
A1: Specify parameters for at least one waveform; such as amplitude, frequency, shape, DC offset, number of repetitions (cycles), and so forth; and identify whether the specified amplitude and shape relates to voltage or to current.
A2: Optionally, create a sequence of digital amplitudes for at least one cycle of the waveform.
A3: Generate at least one cycle of the specified waveform by converting the sequence of digital amplitudes, if so generated in A2, to an analog waveform.
A4: Apply the generated waveform (optionally filtered) to a waveform electrode and a return electrode and thus to the intervening tissue.
A5: Measure a discrete sequence of analog samples of waveform amplitudes of at least one electrical property (such as current flow or voltage) where each sample element of the sequence is acquired at a known time (preferably with the samples separated by known equal time intervals).
A6: Digitize and save the sequence of discrete samples as a sequence of digital numbers.
A7: Optionally, preprocess the sequence, such as by subtracting known interference or bias from each number in the sequence, or apply any calibration adjustments.
A8: If necessary, convert the digital sequence to known engineering units (such as volts or amperes).
A9: Match the digital sequence to a best fit ideal mathematical function, such as a function which is the sum of some constant value, plus a cyclic function such as a sine or cosine function or square wave, plus an exponential decay function asymptotic to zero (where time is the independent variable). That is, determine specific coefficients (parameters) of the terms of such a composite function so that the function closely approximates the sequence of digital values at the times associated with those values.
A10: Derive certain parameters which parameterize the best fit ideal function; such as minimum, maximum, and mean amplitude; frequency of each cyclic term (if any) of the function; phase relationship of each cyclic component to the applied waveform; exponential decay rate (time-constant), and the value at a known time.
A11: Optionally, compute the values of the best fit ideal function at the times corresponding to the numbers in the sample sequence, compare those function values with the digital numbers of the preprocessed sequence, and compute a single statistic (such as the root-mean-square of the differences) that represents the degree of fit or confidence of this procedure.
A12: Derive electrical properties (such as complex impedance) from the parameters characterizing the best fit function.
A13: Optionally, infer best fit through analog methods and calculate electrical parameters such as resistance or capacitance.
A14: Repeat steps A4 through A13 for various other pairs of waveform and return electrodes.
A15: Optionally, repeat steps A1 through A14 for applied waveforms with other specifications (such as other frequencies).
A16: Optionally, map the values of the derived electrical properties for the electrodes to normalized, relative values (e.g., mapping the minimum value to 0.0, the maximum value to 1.0, and all other values linearly positioned between 0 and 1).
A17: Display the actual or positionally represented values for the electrodes in any of several forms, such as bar or bubble charts for the value(s) computed for each electrode, interpolated contour plots related to the values and the electrode locations, or shaded plots related to the electrode array geometry and the computed values.
A18: Optionally, infer nerve location from electrical circuit analog information and display using any of the methods of A17.
A19: Optionally enhance the charted or graphed values by means of zoom, contrast, or threshold controls

Not all the foregoing steps necessarily need to be performed, or be performed in the exact order shown above. For example, steps A7 and A8 may be reversed in their order, and other steps may be incorporated as well.

The aforementioned method steps will now be discussed in more detail.

Step A1 specifies parameters for at least one waveform to be applied between at least one pair of electrodes, referred to as a waveform electrode and a return electrode. These waveform parameters may be fixed, automatic, or user-selected. These parameters may be loaded into the waveform generator 8 by the microprocessor 16. The generator may be capable of generating the waveform as an additive composite of several primitive component waveforms (for example, a constant offset, a repeated cyclic waveform, and an acyclic waveform such as an impulse or exponential decay). The specification parameters may include any or all of the following: the amplitude of a constant DC offset component for the waveform, the frequency of one or more cyclic components of the waveform, the amplitude and shape of each cyclic component over time (such as sinusoidal, triangular, or rectangular cycles as would be viewed when time is plotted on the X axis and the signal is plotted on the Y axis), the number of repetitions of each cyclic component, the amplitude and duration of an impulse, and the initial value and time constant of an exponentially decaying component. Furthermore, if the choice is implemented in the electronics hardware, the microprocessor 16 may specify whether the foregoing specified parameters control a voltage or a current waveform.

Another specification parameter useful to simplify later computation is the number of repetitions of the waveform to generate and apply to the subject's tissue before acquiring samples. A sufficient number of these preconditioning repetitions may charge the capacitance of the subject's tissue to nearly the steady state value—especially if there is a constant offset (DC) component present in the applied waveform.

In optional Step A2, an arbitrary waveform may be generated by digital means by creating a sequence of discrete digital amplitude values. These discrete values may describe a single cycle of a waveform of N cycles or may be the full length of an acyclic waveform.

For example, a sinusoidal waveform may be generated as follows, where the waveform amplitude for the i^{th} sample of the sequence is given by
Gᵢ=G₀[cos(2πn/N+P₀)]+G_{DC}
where
G_{DC} is a constant offset amplitude,
G₀ is the peak amplitude,
P₀ is the initial phase angle for the cycle,
i is the index of a sample, and
N is the number of samples per cycle.
   If G_{DC} is greater than or equal to G₀, then the waveform will be monophasic. If P₀ equals −π radians, then the waveform will begin at its minimum amplitude; the latter is advantageous because there will be minimal discontinuity at the very beginning of the pulse cycle. In an embodiment of the present invention, all the samples are generated at regular time intervals, so that the i^{th} sample represents amplitude at time t=i·T, where T is the period of the regular time intervals.

Step A3 generates at least one repetition (cycle) of the waveform according to the specified parameters. If Step A2 was employed to generate the waveform digitally, then the digital samples are passed at a given rate to a digital-to-analog converter (DAC) to generate a continuous analog voltage or current waveform. Filter circuit components, such as a passive filter, may be used to smooth the shape of the applied waveform, especially if the waveform is intended to be a sinusoidal or other waveform devoid of sudden (e.g., step change) transitions.

Step A4 applies the generated waveform to a pair of electrodes, one electrode being a waveform electrode and the other a return electrode. The electrodes in contact with the surface of the subject's tissue thereby apply the waveform to the intervening tissue. If the generator controls the voltage of the applied electrical waveform, then the electrical characteristics or properties of the tissue affect the measurable current flow attributes of the measured waveform. Such electrical characteristics or properties generally affect the amplitude and phase angle of the waveform. Conversely, if the generator controls the current of the applied electrical waveform, then the electrical characteristics or properties of the tissue affect the measurable voltage attributes of the measured waveform. It is worth noting that safety circuits may be included to prevent excessive current from being applied to the subject.

Step A5 acquires and measures a sequence of discrete analog samples of waveform amplitudes of at least one electrical property. The sample elements W[i] of the sequence W are taken at known times, preferably separated by a constant, known time interval T. The electrical property measured directly between the two electrodes is current flow, voltage differential, or both. If a controlled voltage waveform is applied, then at least the current flow waveform W would normally be sampled. If a controlled current waveform is applied, then at least the voltage differential waveform W would normally be sampled.

Acquiring sufficiently many samples (such as at least 20 per cycle for a periodic waveform) allows the microprocessor 16 to perform a best fit match of the acquired samples with a parameterized mathematical function representing an ideal waveform as explained in more detail below. From the parameters characterizing the best fit mathematical function or waveform, the microprocessor 16 can derive various numeric electrical characteristics or properties of the tissue, such as impedance, admittance, resistance, or capacitance, as will be described below. Alternatively, if the generator controls the current flow of the applied electrical waveform, then the electrical characteristics or properties of the tissue affect the waveform of the voltage that is required to cause the specified current to flow. In that case, sufficiently many voltage attribute samples are acquired to yield sufficient data for curve fitting and statistical purposes, and an ideal waveform is fit optimally to the data.

Step A6 digitizes the sequence of acquired discrete samples as a sequence of digital numbers and saves the numeric sequence (which can also be called W) in memory for further processing. This step uses a conventional analog-to digital-converter (ADC) circuit or digital signal processor (DSP) of adequate speed, range, and resolution (preferably at least 12 bits) to convert the measured sample to digital values as is well known in the art. For the case of measuring current flow, the conventional approach employs a precision resistor in series with the tissue to measure the voltage differential across the resistor as current flows through the tissue and therefore through the resistor. By applying Ohm's law, the instantaneous current flow Iᵢ becomes Iᵢ=Vᵢ/R_{M}, where R_{M} is the known resistance of the precision resistor and Vᵢ is the instantaneous voltage corresponding to the i^{th} sample time. For simplicity and clarity, R_{M} may be ignored and, in effect, be subsumed within the current measuring means M_{I} (see FIG. 1) and appropriately compensated for in the following calculations. Alternatively, the voltage can be measured on the sample side of the sense resistor; compensation would thereby be avoided.

Step A7 is optional, but may be required to preprocess the sequence of discrete measurements in order to yield an acceptable sequence. For example, a known bias voltage may have been added within the circuitry (such as to insure that all measurements are non-negative). In an embodiment, the known bias may be subtracted from each measurement sample before proceeding. A more sophisticated embodiment of the invention might apply the waveform across a precision calibration circuit before applying the same waveform to real tissue. This may be used to determine characteristics of the internal measuring circuitry itself, such as leakage current through the ADC and the multiplexer 10. The microprocessor 16 may remove these effects from the digital value of each sample, such as by subtraction. Another embodiment of preprocessing involves removing noise from the sequence by applying known signal processing techniques, such as boxcar or Gaussian averaging. Another example embodiment of preprocessing applies multiplicative or additive adjustments from an experimentally created calibration table or polynomial function. This process may be used to remove any nonlinearity due to the electronic circuitry.

Typically, the binary integer numeric values output by the ADC in Step A6 (and optionally preprocessed in Step A7) will not correspond to standard engineering units. For this reason, Step A8 involves converting the numeric data to known engineering units, such as volts or amperes, as appropriate. The conversion may comprise multiplication by a known factor or a calibrated conversion factor, the latter of which depends directly on the reference voltage used with the ADC and the properties of the ADC circuit. An example multiplication factor is negative one (−1) to invert the results, which may present the values in a more useful format or improve their presentation to a user (such as presenting the inverse of impedance so that nerves are indicated by peaks instead of valleys). A constant offset value might also need to be added. The value of such an offset may be determined by a calibration scheme or by computation based on the ADC circuit design and components. For purposes of this description, the sequence of preprocessed, converted, and calibrated values is denoted as W′.

Step A9 determines the coefficients of a mathematical function F(t) which provides a best fit approximation match to the digital numeric sequence W′ at time t=iT. Normally, the mathematical function may be chosen out of a set of parameterized functions which differ only by the values of a small number of parameters or coefficients. The independent variable of the function may be time t or a unit related to time (e.g., clock cycles, sample numbers, etc.). Such a function may be the composite (sum or product) of several simpler component or basis functions with the same independent variable t. These may comprise a constant amplitude value; one or more periodic (cyclic) functions such as a conventional sine function, cosine function, square wave; and/or an exponential decay function asymptotic to zero. These component functions are consistent with the electrical characteristics expected of a parallel RC circuit such as that illustrated in FIG. 6. For example, a constant amplitude value reflects an offset (e.g., direct current) component of an applied waveform, the cyclic function reflects the cyclic nature of the applied waveform, and the decay function reflects the capacitive nature of tissue, including nerve tissue, in the presence of an electric field. Thus, Step A10 derives estimates for specific parameters of the terms of such a composite mathematical function so that the resulting function closely approximates (i.e., forms a best fit approximation for) the sequence of digital values at the times associated with those values.

For example, a suitable mathematical function may be:
F(t)=A_{DC}+A_{AC}[ cos(2πt/(NT)+P₀)]+A₀[ exp(−A_{RC}t)]
where
A_{DC} is the amplitude of the constant direct current component,
A_{AC} is the amplitude of the periodic component,
A₀ is the amplitude of the decay component, and
A_{RC} is the decay rate constant.

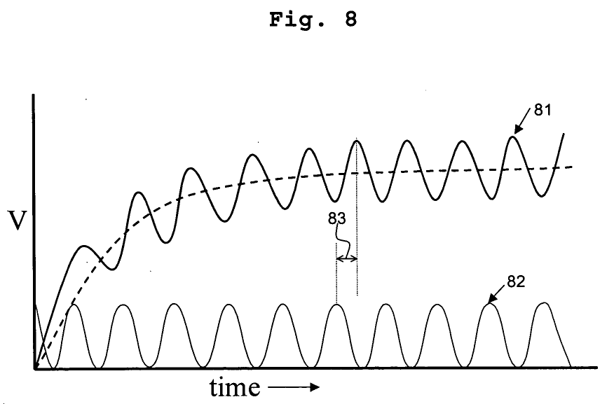

When the applied waveform is time varying, e.g., a sine or square wave, the time varying nature of the detected current or voltage provides information about the underlying tissue. For example, comparing the time varying measured waveform to the applied waveform may provide phase relationship information, e.g., the phase shift of the measured waveform compared to the applied waveform. An example of phase shift that may be detected is illustrated in FIG. 8, which shows an idealized measured waveform 81 in the presence of the zero-biased sine wave applied waveform 82. Referring to FIG. 8, the phase shift refers to the delay 83 in the peaks of the measured waveform compared to the applied waveform 82. Since the phase shift is related to the capacitive characteristics or properties of underlying tissue, the phase relationship of the measured waveform can be useful in discriminating tissue types.

One method of estimating the parameters of the ideal composite function is to represent the preprocessed sample sequence W′ as the sample by sample sum of several component sequences, which correspond to the component functions. For example, there may be a component sequence of constant values, a periodic component sequence corresponding to discrete values of a cyclic function, and a component sequence corresponding to discrete values of an exponential decay. A sample by sample sum of these functions would equal the samples of the preprocessed numeric sample sequence. Such a composite function may thus be represented as
W′[i]=W_{DC}[i]+W_{AC}[i]+W_{decay}[i]
where
W_{DC}[i]=C_{DC},
W_{AC}[i]=C_{AC}[ cos(2πi/N)+Cₚₕₐₛₑ],
W_{decay}[i]=C₀[ exp(−C_{RC}iT), and
C_{DC}, C_{AC}, Cₚₕₐₛₑ, C₀, and C_{RC} are parametric constants.

One method for constructing an approximation to the periodic component sequence W_{AC} is to construct an intermediate aperiodic component sequence Wᵢₙₜₑᵣₘ as follows. Let the i^{th} element of the intermediate sequence be the average of exactly one cycle of samples where the cycle is approximately centered on the i^{th} element. This will not define the first ½ and the last ½ cycle of samples in the intermediate sequence Wᵢₙₜₑᵣₘ, but portions can be set to W′ or simply ignored. It may be assumed that at least several cycles of preprocessed data were acquired so that the intermediate sequence contains at least one cycle of well defined data. Here cycle means the equivalent of one cycle of the cyclic component of the applied waveform. Next, the intermediate sequence is subtracted from the preprocessed sequence (ignoring the first ½ cycle and last ½ cycle) to yield
W_{AC}[i]≈W′[i]−Wᵢₙₜₑᵣₘ[i].
The result W_{AC} will be an approximation of the cyclic sequence component of the preprocessed sample sequence. This approximation may then be correlated with the cosine and sine functions having the same period and phase as the applied waveform as given by
C_{AC1}=2Σᵢ(W_{AC}[i])[ cos(2πi/N)], and
C_{ACj}=2Σᵢ(W_{AC}[i])[ sin(2πi/N)],
where
N is the number of samples acquired per cycle of the periodic component of the applied waveform,
C_{AC1} is the real part of the periodic amplitude expressed as a complex number, and
C_{ACj} is the imaginary part of the periodic amplitude expressed as a complex number.
   Alternatively, the periodic amplitude may be represented by a non-negative amplitude C_{AC} and an angle P₀ as given by
   C_{AC}=\sqrt{(C_{AC1}²+C_{ACj}²)} and
   P₀=arctan (C_{ACj}/C_{AC1}).
   Note that this approximation works for both a periodic (preferably sinusoidal) voltage function and a periodic current function. When this method is implemented to determine C_{AC} and P₀, the approximation W_{AC}[i]≅W′[i]−Wᵢₙₜₑᵣₘ[i] is used in the formulae for C_{AC1} and C_{ACj}

The constant amplitude component W_{DC} may be estimated by averaging the values of the samples that were collected over exactly an integral number of cycles (assuming that the applied waveform has a cyclic component as for a sinusoidal waveform). Alternatively, W_{DC} may be set to the average of the last portion of Wᵢₙₜₑᵣₘ. If there is substantial capacitance in the tissue, there will be a measurable exponential decay component W_{decay}, so that the average should be taken only over the last few cycles, after which presumably most of the decay will have already occurred. Either sufficient numbers of samples should be taken for this to be true, or there should be sufficient preconditioning repetitions of the applied waveform before acquiring the data, as described above.

Step A10 derives the constants which parameterize the best fit ideal function F(t). These may be the parametric constants C_{DC}, C_{AC}, Cₚₕₐₛₑ, C₀, and C_{RC} in the foregoing example, or they may include other parametric constants such as the minimum and maximum amplitude, the phase, the duty cycle, and the frequency of a rectangular function. Step A10 may simply select the constants C_{DC}, C_{AC}, Cₚₕₐₛₑ, C₀, and C_{RC}. Preferably, these values may be refined by an iterative optimization procedure by minimizing the mismatch between W′ and F using well known optimization techniques. The resulting refined, optimized values A_{DC}, A_{AC}, Aₚₕₐₛₑ, A₀, and A_{RC} for the constants C_{DC}, C_{AC}, Cₚₕₐₛₑ, C₀, and C_{RC}, respectively, optimally parameterize the composite ideal function F(t) that best approximates W′ as given by
F(t)=A_{DC}+A_{AC}[ cos(2πt/(NT)+P₀)]+A₀[ exp(−A_{RC}t)].
Note that this is only an example of a single embodiment; and other approximating composite functions may be used in addition to or instead of those above example as would be known to one of skill in the art.

Step A11 is optional and computes the values F(iT) of the best fit ideal function at the times iT, which correspond to the numeric samples in the sample sequence. Step A12 then compares those function values F(iT) with the digital numbers W′[i] of the preprocessed sequence W′. Finally, optional Step A12 computes a single statistic (such as the root-mean-square of the differences) which represents the degree of deviation or lack of confidence of this procedure as given by
RMS(F, W′)={square root over (Σᵢ(F(iT)−W′[i])²)}

Using the parametric constants which characterize the best fit function F(t) which matches W′, Step A12 derives electrical properties of the tissue. The electrical properties of interest might include any of impedance, admittance, resistance, susceptance, capacitance, or phase shift, for example.

Regardless whether the voltage or the current is the controlled property of the electrical waveform applied to the tissue, when using a sinusoidal waveform one can apply the complex form of Ohm's Law to find the complex impedance Z of the tissue by
Z=V/I
where
Z is the impedance (with real resistive and imaginary reactive components),
V is the periodic component of the voltage waveform (either applied or measured),
I is the periodic component of the current waveform (either applied or measured), and where complex quantities V and I are measured with respect to the same phase reference (i.e., synchronous cosine and sine references). If it is assumed that the parameters A_{AC} and P₀ are known for each of the component functions approximating cyclic voltage waveform and current waveform, then the complex impedance Z is:
   Z=(V_{AC}/I_{AC})cos(V_{P}−I_{P})+j(V_{AC}/I_{AC})sin(V_{P}−I_{P})
   where
V_{AC} is the amplitude of the periodic voltage component,
V_{P} is the phase angle of the voltage component,
I_{AC} is the amplitude of the periodic current component,
I_{P} is the phase angle of the current component, and
   j=\sqrt{(−1)}
   Furthermore, the complex admittance Y is given by this complex division:
   Y=1/Z.
   The real and imaginary components of Y are the conductance and susceptance, respectively. Further, the tissue resistance R and the tissue capacitance C are given by:
   R=1/real(Y);
   C=imag(Y)/(2πF)
   where
F is the frequency of the applied periodic component,
real(Y) is the real part of Y, and
imag(Y) is the imaginary part of Y.

The following are several alternative methods of computing the resistance and capacitance of the tissue for Step 13 using the values of various of the parametric constants A_{DC}, A_{AC}, Aₚₕₐₛₑ, A₀, and A_{RC} computed above.

First, if there is a non-zero constant offset (DC) voltage component V_{DC} in the voltage waveform and a non-zero constant offset current component I_{DC} in the current waveform, then the tissue resistance R can be computed as
R=V_{DC}/I_{DC}
using Ohm's Law. This has been found to be more accurate than using the previous formula for R in a prototype of an embodiment of the present invention.

Second, there is an alternative method of computing C if there is a substantial, measurable exponential decay component W_{decay} and therefore a matching decay component of F(t), namely A₀ exp(−A_{RC}t). In this circumstance, the RC time constant is
C_{RC}=1/A_{RC}.
Furthermore,
C=C_{RC}/R.

In the case where nerve tissue is modeled as a bulk parallel RC circuit, one can obtain R and C for a controlled sinusoidal voltage waveform as follows:

$$\begin{matrix}R = - {R}_{M} ( {V}_{M \text{-} max} + {V}_{M \text{-} min} + {V}_{A \text{-} peak} ) / ( {V}_{M \text{-} max} + {V}_{M \text{-} min} ) ; \\ C = 2 {V}_{A \text{-} peak} \frac{\sqrt{[ \vert ( {V}_{M \text{-} min} {V}_{M \text{-} max} ) / ( {V}_{A \text{-} peak}^{2} - {V}_{M \text{-} pp}^{2} ) \vert ]}}{[ π {FR}_{M} ( {V}_{M \text{-} max} + {V}_{M \text{-} min} + {V}_{A \text{-} peak} ) ]}\end{matrix}$$

where
R_{M} is the resistance across the sense resistor,
V_{M-min} is the minimum measured voltage across R_{M},
V_{M-max} is the maximum measured voltage across R_{M},
V_{A-peak} is the maximum applied voltage,
V_{M-pp}=V_{M-max}−V_{M-min}, and
F is the frequency of the sinusoidal periodic waveform W_{AC}.

Modeling the nerve tissue as a bulk parallel RC circuit, another alternative formulation for determining R and C for a controlled current waveform is:

$$\begin{matrix}R = ( {V}_{T \text{-} max} + {V}_{T \text{-} min} - 2 {V}_{r} ) / \overline{I} \\ C = \frac{\overline{I} \sqrt{\vert ( {V}_{T \text{-} max} - {V}_{r} ) ( {V}_{T \text{-} min} - {V}_{r} ) \vert}}{π F ( {V}_{T \text{-} max} - {V}_{T \text{-} min} ) ( {V}_{T \text{-} max} + {V}_{T \text{-} min} - 2 {V}_{r} )}\end{matrix}$$

where
V_{T-min} is the minimum measured voltage across the tissue at steady state,
V_{T-max} is the maximum measured voltage across the tissue at steady state,
Vᵣ is the rest potential across the tissue,
\overline{I} is the controlled current amplitude, and
F is the frequency of the sinusoidal periodic waveform W_{AC}.

As an optional alternative, in Step A13, the best fit parameters described above may be inferred through analog methods, such as using analog circuit elements. Using analog derived parameters, the electrical characteristics, such as resistance and capacitance, may also be determined.

Optional Step A14 involves repeating Steps A4 through A13 for various other pairs of waveform and return electrodes, if there are any. This step includes saving the measured and computed electrical properties of the tissue for each electrode pair and associating them with the geometrical tissue-contact locations of the electrodes involved.

Optional Step A15 involves repeating Steps A1 through A14 for applied waveforms with other specifications, if there are any. For example, Steps A1 through A14 might apply waveforms of each of two or more frequencies, or two or more waveform shapes, or two or more amplitudes, or combinations thereof. Differing results for differing waveforms can be used to verify or more accurately compute some tissue property. For example, susceptance would be expected to increase (more or less) linearly with frequency, while resistance would not.

Optional Step A16 maps the values for the electrodes to normalized, relative values in preparation for certain kinds of graphical plots. Also, this step may be used to simplify the differentiation between types of underlying tissue (such as nerve versus non-nerve tissue). This step may also remove the effects of certain irrelevant properties of the data (such as lower overall impedances due to tissue hydration or the mean distance between waveform and return electrodes). In a preferred embodiment, this step maps the minimum value of the tissue property of interest to 0.0, the maximum value to 1.0, and all other values to somewhere between 0 and 1 (e.g., linearly interpolated).

Step A17 displays the actual or normalized values for the electrodes in any of several graphic forms. Graphic forms may include, but are not limited to: bar or bubble charts for the value or values computed for each of electrodes; interpolated contour plots related to the values and the electrode locations; shaded plots related to the electrode array geometry; and the computed values. Computerized charting and spreadsheet programs offer these and many other exemplary formats for graphically presenting numeric data in graphical form—any of which could be used. Formats that render 3-D (or higher dimensionality) data on a graphical screen may be the preferred. This is because the electrode locations on tissue surface may be depicted as X and Y coordinates (measured in millimeters for example) and at least one tissue property may be treated as a Z coordinate axis (such as the admittance in siemens). More than one tissue property may be plotted as side-by-side graphs or even overlaid graphs.

In Step A17, a combination of graphic forms may be generated and presented simultaneous. For example, one property may be displayed as a contour graph while the same or another property may be displayed as numbers at locations corresponding to the electrode locations on a scaled pictorial representation of the electrode array.

As a further option, in Step A18, the mapping of measured and calculated values to electrodes may be used to infer nerve locations, which are typically indicated by local impedance minimums. The inferred locations of nerves may then be displayed using any of the techniques described for Step A17.

Optional Step A19 provides control of further visual enhancement of the charted or graphed values. This step may comprise the provision and use of interactive controls which allow an operator to rotate or zoom in on the data, or which adjust contrast, coloration, or thresholding of the graphical rendering. Furthermore, a global or local nonlinear function may be applied to the actual or normalized values of the plotted characteristic, for display enhancement, to exaggerate local maxima or minima of the plotted characteristic, for example.

The interpretation of the results of Steps 17, 18 or 19 may be left to a human operator or may be automated by highlighting regions of interest—for example electrodes above (or below) an absolute or relative threshold value. Another example would be highlighting local maxima (or minima) or connected ridges or saddles on smoothed and interpolated contour plots of the data.

Not all the Steps need to be performed exactly in the order described above. For example, optional Step 12 may be performed later (if at all), if the data it requires are kept available.

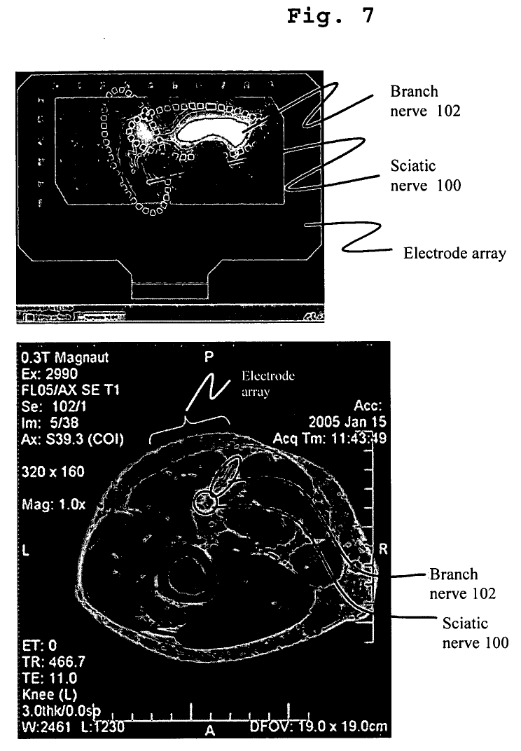

FIG. 7 shows one such graphical display of data taken from the region over the sciatic nerve 100 where a major branch 102 originates. The accompanying MRI slice depicts the same region rotated 90° to the plane of the graphical image.

The methods of the present invention may further comprise the steps of generating a graphical image. The image would relate to the measured or calculated values associated with the electrodes, reflect certain electrical properties of the underlying tissue, and presumably indicate the presence of underlying tissue (such as nerves) having distinguishing electrical properties.

A computer readable medium embodying the present invention may carry instructions to cause a processor to institute the performance of the above method or a variation thereof, and may optionally incorporate none, some, or all of the optional steps.

The foregoing description of various embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments and with various modifications as are suited to the particular use contemplated.

We claim:

1. A system for discriminating tissue, comprising:
a processor;
a waveform generator configured to generate an applied waveform at least one property of which is specified by the processor;
a waveform electrode and a return electrode coupled to the waveform generator and suitable for application to skin of a subject;
a measurement circuit coupled to the processor, the waveform electrode and the return electrode and configured to measure at least one electrical attribute between the electrodes; and
a display coupled to the processor;
wherein the processor is programmed to:
(a) specify parameters to the waveform generator describing at least one waveform and whether the waveform relates to an applied voltage or an applied current;
(b) direct the waveform generator to generate at least one repetition of the waveform which is applied across the waveform electrode and the return electrode when each is positioned on the skin of a person;
(c) receive a temporally discrete sequence of samples of at least one electrical property measured between the waveform electrode and the return electrode;
(d) save the discrete sequence of samples as a sequence of digital numbers;
(e) match the sequence of digital numbers to a mathematical function having an independent variable of time, so that the function approximates the sequence of digital values at the times associated with those values;
(f) derive electrical properties from the parameters characterizing the mathematical function; and
(g) present the derived electrical property values in a human understandable form.

2. The system of claim 1, wherein one specified waveform parameter is amplitude.

3. The system of claim 1, wherein one specified waveform parameter is frequency.

4. The system of claim 1, wherein the specified waveform parameter is a shape of the waveform.

5. The system of claim 4, wherein the shape of the waveform is sinusoidal plus a constant offset value.

6. The system of claim 4, wherein the shape of the waveform is rectangular.

7. The system of claim 1, wherein the waveform is specified by a discrete sequence of digital amplitude values for at least one repetition of the waveform, where each digital amplitude value corresponds to a time within the duration period of the waveform.

8. The system of claim 7, wherein the waveform is filtered to convert the sequence of discrete digital amplitudes into a substantially smooth continuous waveform.

9. The system of claim 1, wherein the waveform is generated for a specified number of cycles.

10. The system of claim 1, wherein the measured electrical property is current flow.

11. The system of claim 1, wherein the measured electrical property is voltage.

12. The system of claim 1, wherein the sequence of samples is acquired at a rate sufficient to characterize a temporal change of the measured property relative to the applied waveform.

13. The system of claim 1, wherein at least 8 samples are acquired per cycle of a cyclic component of the applied waveform.

14. The system of claim 1, wherein the samples are acquired at a substantially constant frequency.

15. The system of claim 1, wherein the digital numbers are values in an engineering unit.

16. The system of claim 15, wherein the units are volts.

17. The system of claim 15, wherein the units are amperes.

18. The system of claim 1, wherein values of the sequence of samples are preprocessed to adjust the values to accomplish any subset of the following: subtract off a known bias from each value, remove interference and noise, and apply a calibration correction.

19. The system of claim 1, wherein the mathematical function comprises:
a composite of a constant value,
a cyclic function such as a sine or cosine function or square wave, and
an exponential decay function asymptotic to zero, where time is an independent variable.

20. The system of claim 19, wherein the mathematical function may be fully specified by a few specific parametric coefficients, where the number of coefficients is substantially less than the number of samples in an acquired sequence.

21. The system of claim 20, wherein the coefficients of the mathematical function may include one or an arithmetic combination of the following: minimum amplitude, maximum amplitude, mean amplitude, frequency of each cyclic component function, and phase relationship to the applied waveform.

22. The system of claim 20, wherein the coefficients of an acyclic exponential decay component of the mathematical function are a time-constant and a value at a known time.

23. The system of claim 1, wherein the processor is further programmed to:
compute values of the mathematical function at times corresponding to individual values in the sequence of samples;
compare the calculated function values with the values of the sequence of samples; and
compute a statistic which represents a degree of fit between the sequence of samples and the mathematical function.

24. The system of claim 23, wherein the statistic is a root-mean-square (RMS) of a difference between the sequence of sample values and the corresponding calculated function values.

25. The system of claim 1, wherein the derived electrical properties include one or more of impedance, admittance, resistance, permittivity, capacitance and phase angle.

26. The system of claim 1, wherein the processor is further programmed to repeat the procedural steps for a second waveform having a different parameter.

27. The system of claim 26, wherein the differing parameter is frequency.

28. The system of claim 26, wherein the differing parameter is amplitude.

29. The system of claim 1, wherein each of the sequence of digital numbers is normalized.

30. The system of claim 1, wherein the human understandable form comprises a graphical presentation of at least one of the following types: bar chart for property values computed for the electrodes, bubble chart, interpolated contour plot relating values to locations of corresponding electrodes, and shaded plot relating electrode array geometry and computed values.

31. The system of claim 1, wherein derived electrical property values of the human understandable form are presented in a graphical form

32. The system of claim 31, wherein the graphical form includes an enhancement means chosen from the following: a zoom control for local magnification of a part of the graph, a contrast enhancement, a threshold control, a nonlinear exaggeration of values within some range, and a highlighting of local maxima and connected regions of local maxima.

33. The system of claim 1, wherein the human understandable form is a table of numbers expressing the property values.

34. A method for discriminating tissue within a subject, comprising:
(a) specifying parameters describing at least one waveform and specifying whether the waveform relates to an applied voltage or an applied current;
(b) generating at least one repetition of the specified waveform;
(c) applying the generated waveform across a waveform electrode and a return electrode and thereby to intervening tissue of the subject with which the electrodes are in contact;
(d) measuring a temporally discrete sequence of analog samples of at least one electrical property;
(e) saving the sequence of discrete samples as a sequence of digital numbers;
(f) matching the sequence of digital numbers to a mathematical function having time as an independent variable so that the mathematical function approximates the sequence of digital numbers;
(g) deriving electrical property values from parameters characterizing the mathematical function;
(h) repeating steps b through g for various other pairs of waveform and return electrodes; and
(i) presenting the derived electrical property values in a human understandable form.

35. The method of claim 34, wherein one waveform parameter is amplitude.

36. The method of claim 34, wherein one waveform parameter is frequency.

37. The method of claim 34, wherein the waveform parameter is a shape of the waveform.

38. The method of claim 37, wherein the shape of the waveform is sinusoidal plus a constant offset value.

39. The method of claim 37, wherein the shape of the waveform is rectangular.

40. The method of claim 34, wherein the waveform is specified by a discrete sequence of digital amplitude values for at least one repetition of the waveform, where each digital amplitude value corresponds to a time within the duration period of the waveform.

41. The method of claim 40, wherein the waveform is filtered to convert the sequence of discrete digital amplitudes into a substantially smooth continuous waveform.

42. The method of claim 34, wherein the waveform is cyclically repeated for a given number of repetitions.

43. The method of claim 34, wherein the measured electrical property is the immediate, present current flow.

44. The method of claim 34, wherein the measured electrical property is the immediate, present voltage.

45. The method of claim 34, wherein the sequence of samples is acquired at a rate sufficient to characterize the temporal change of the measured property relative to the applied waveform.

46. The method of claim 34, wherein at least 8 samples are acquired per cycle of a cyclic component of the applied waveform.

47. The method of claim 34, wherein the samples are acquired at an essentially constant rate.

48. The method of claim 34, wherein the samples of each measured property are digital numbers.

49. The method of claim 48, wherein the digital numbers are values in a standard engineering unit.

50. The method of claim 49, wherein the property is voltage and the units are volts.

51. The method of claim 49, wherein the property is current and the units are amperes.

52. The method of claim 34, wherein the values of the sequence of samples are preprocessed to adjust the values for any subset of the following reasons: to subtract off a known bias from each value, to remove interference and noise, or to apply a calibration correction.

53. The method of claim 34, wherein the mathematical function is the composite of some constant value, plus a cyclic function such as a sine or cosine function or square wave, plus an exponential decay function asymptotic to zero, where time is the independent variable.

54. The method of claim 53, wherein the mathematical function may be fully specified by a few specific parametric coefficients, where the number of coefficients is substantially less than the number of samples in an acquired sequence.

55. The method of claim 54, wherein the coefficients of the mathematical function may include some subset of or arithmetic combination of the following: minimum amplitude, maximum amplitude, mean amplitude, the frequency of each cyclic component function, and its phase relationship to the applied waveform.

56. The method of claim 54, wherein the coefficients of an acyclic exponential decay component of the ideal function are its time-constant and its value at a known time, such as the starting time of the applied waveform.

57. The method of claim 34, wherein the method includes an additional step which computes the values of the mathematical function at the times corresponding to the values in the sample sequence; compares those function values with the values of the sequence of samples; and computes a single statistic which represents a degree of fit between the sequence of samples and the mathematical function.

58. The method of claim 57, wherein the single statistic is the root-mean-square (RMS) of the differences between the sample values and the corresponding values of the mathematical function.

59. The method of claim 34, wherein the derived electrical properties include one or more properties from the following: impedance, admittance, resistance, permittivity, capacitance or phase angle.

60. The method of claim 34, wherein the method steps are applied to at least one pair of electrodes consisting of a waveform electrode and a return electrode.

61. The method of claim 34, wherein the method steps are repeated for applied waveforms with specifications differing from previous waveforms;

62. The method of claim 61, wherein a differing specification is frequency.

63. The method of claim 61, wherein a differing specification is amplitude.

64. The method of claim 34, wherein the properties for the various electrodes are normalized to relative values.

65. The method of claim 64, wherein the normalized values are in the range of 0.0 to 1.0, where the minimum of all the property values is normalized to 0.0, the maximum value is normalized to 1.0, and all other value of the property are normalized to values linearly interpolated between 0.0 and 1.0.

66. The method of claim 34, wherein the human understandable form includes a graphical presentation using at least one of the following choices: a bar chart for property values computed for the electrodes, a bubble chart relating values and locations of the electrodes, interpolated contour plots relating values and locations of the electrodes, and shaded plots relating electrode array geometry and computed values.

67. The method of claim 34, wherein property values of the human understandable form are in a graphical form

68. The method of claim 67, wherein the graph is enhanced by at least one enhancement control chosen from the following: zoom control for local magnification of part of the graph, contrast enhancement, a threshold control, a nonlinear exaggeration of values within some range, and highlighting of local maxima and connected regions of local maxima.

69. The method of claim 34, wherein the human understandable form is a table of numbers expressing the property values.

70. The method of claim 34, wherein a discriminated tissue is nerve tissue.
